(19) United States
(12) Patent Application Publication
(10) Pub. No.: US 2009/0062739 A1
(43) Pub. Date: Mar. 5, 2009

(54) Catheter Guidewire Tracking System and Method

(75) Inventor: Peter Traneus Anderson, Andover, MA (US)

Correspondence Address: GE HEALTHCARE;c/o FLETCHER YODER, PC, P.O. BOX 692289, HOUSTON, TX, 77269-2289, US

(73) Assignee: General Electric Company, Schenectady, NY (US)

(21) Appl. No.: 11/848,796

(22) Filed: Aug. 31, 2007

(51) Int. Cl. A61M 25/09; A61M 25/095

(52) U.S. Cl. 604/164.13; 604/538

(57) ABSTRACT

Provided in certain embodiments is a tracking system, having a transformer-coupled guidewire system, having a coil comprising a primary winding, configured to enable a guidewire including a secondary winding to be inductively coupled to the primary winding, and configured to enable a catheter to be slid over the guidewire while the secondary winding is inductively coupled to the primary winding. Also provided is a tracking method, including positioning a primary winding about a first coil that is integral to a first end of a guidewire, inductively coupling the primary winding and the first coil, transferring the energy between the first coil and a second coil integral to a second end of the guidewire, passing a catheter over the guidewire, and tracking the guidewire while the catheter is slid over the guidewire.

### BACKGROUND

The present invention generally relates to imaging and image-guided navigation. In particular, the present invention relates to a system and method of tracking a guidewire and/or catheter.

Medical practitioners, such as doctors, surgeons, and other medical professionals, often rely upon technology when performing medical procedures, including image-guided surgery or examination. For instance, during image-guided procedures, an instrument tracking or navigation system may provide positioning information of a medical instrument with respect to the patient or a reference coordinate system. The tracking or navigation system enables the medical practitioner to visualize the patient's anatomy and track the relative position and orientation of the instrument. Thus, a medical practitioner may refer to the tracking system to ascertain the position of the medical instrument when the instrument is not within the practitioner's line of sight, and enable a determination when the instrument is positioned in a desired location. Accordingly, the medical practitioner may locate and operate on a specific area while avoiding other structures. Increased precision in locating medical instruments within a patient may provide for a less invasive medical procedure and also reduce risks associated with more invasive procedures.

In image-guided surgery, the system may display an image positioned in a surgeon's field of view, e.g., such as a selected CT (computed tomography) images, MRI (magnetic resonance images), and several x-ray or fluoroscopic views taken from different angles. Image-guided surgery is of a special utility in surgical procedures, such as brain surgery, arthroscopic procedures on the knee, wrist, shoulder or spine, as well as certain types of angiography, cardiac procedures, interventional radiology and biopsies. In intraoperative or perioperative imaging, images are formed of a region of a patient's body and may be used to track the instrument in relation to a reference coordinate system to aid in an ongoing procedure with a surgical instrument applied to the patient. Further, three-dimensional diagnostic images typically have a spatial resolution that is both rectilinear and accurate to within a very small tolerance, such as to within one millimeter or less. Thus, in tracking and navigation systems, the display may show an image of a surgical tool, biopsy instrument, pedicle screw, probe or other device projected onto a fluoroscopic or other image and enable the surgeon to visualize the orientation of the surgical instrument in relation to the imaged patient's anatomy.

Several medical operations that benefit from image-guided surgery involve very precise planning and control for placement of an elongated probe or other article in a vein, tissue or bone that is internal or difficult to view directly. For example, certain procedures include the insertion of a guidewire and catheter into a patient. Further, for brain surgery, stereotactic frames that define an entry point, probe angle and probe depth are used to access a site in the brain, generally in conjunction with previously compiled three-dimensional diagnostic images, such as MRI, PET (positron emission tomography) or CT scan images. Such systems are also useful for placement of devices (e.g., pedicle screws in the spine), where visual and fluoroscopic imaging directions may not capture a desired view (e.g., an insertion path in bone).

In medical operations that include the insertion of a guidewire or catheter, tracking is beneficial to determine the location of the guidewire or catheter to ensure the instrument is properly located within a region of the patient. Thus, a signal provided to a transmitter or receiver in the guidewire or catheter may enable tracking of the guidewire or catheter as it is threaded into the patient. However, patient safety requirements generally mandate that the patient be isolated from electricity of instruments and other devices. Thus, patient safety requirements generally mandate that electricity provided for tracking or other purposes be isolated from the patient. Further, regulations generally require that devices used proximate or internal to a patient be sterilized to preserve a hygienic operating environment. Thus, a connection to the tracking transmitter, receiver, or other powered device of the catheter should remain sterile.

Accordingly, there is a desire for an improved method for supplying power to a tracking device, such as a guidewire system. Further, there is a need for a sterile system and method for providing power to a guidewire or other tracking system in a hygienic environment.

### BRIEF DESCRIPTION

In accordance with an aspect, provided is a tracking system, comprising a transformer comprising a primary winding, a guidewire comprising a guidewire body, a first coil disposed in a first end of the guidewire body, and a second coil disposed in a second end of the guidewire body, wherein the second coil is inductively coupleable to the primary winding and a catheter disposable over the guidewire while the second coil remains inductively coupled to the primary winding.

In accordance with another aspect, provided is a transformer-coupled guidewire system, comprising a coil comprising a primary winding, configured to enable a guidewire including a secondary winding to be inductively coupled to the primary winding, and configured to enable a catheter to be slid over the guidewire while the secondary winding is inductively coupled to the primary winding.

In accordance with yet another aspect, provided is a tracking method, comprising positioning a primary winding about a first coil that is integral to a first end of a guidewire, inductively coupling the primary winding and the first coil, transferring the energy between the first coil and a second coil integral to a second end of the guidewire, passing a catheter over the guidewire, and tracking the guidewire while the catheter is slid over the guidewire.

In accordance with yet another aspect, provided is an inductively coupled guidewire, comprising a primary coil, and a guidewire mechanically separated from the primary coil and comprising a body, a first coil embedded in a first end of the guidewire, and a second coil embedded in a second end of the guidewire, the first coil and the second coil being electrically coupled.

In accordance with a further aspect, provided is a tracking system, comprising a transformer comprising a primary winding, a guidewire comprising a guidewire body, a first coil disposed in a first end of the guidewire body, and a second coil disposed in a second end of the guidewire body, wherein the second coil is inductively coupleable to the primary winding, a catheter disposable over the guidewire while the second coil remains inductively coupled to the primary winding, and tracker electronics.

### DRAWINGS

These and other features, aspects, and advantages of the present invention will become better understood when the following detailed description is read with reference to the accompanying drawings in which like characters represent like parts throughout the drawings, wherein:

FIG. 1 illustrates a guidewire tracking system in accordance with an embodiment of the present technique;

FIG. 2 illustrates an alternate embodiment of the guidewire tracking system in accordance with an embodiment of the present technique;

FIG. 3 illustrates a flow diagram for a method for tracking a position of an instrument used in accordance with an embodiment of the present technique;

FIG. 4 illustrates the guidewire system with a transformer in accordance with an embodiment of the present technique;

FIG. 5 illustrates the guidewire system with a solenoidal coil in accordance with an embodiment of the present invention;

FIG. 6 illustrated the guidewire system with a catheter slid over the guidewire in accordance with an embodiment of the present technique;

FIGS. 7-8 illustrate the guidewire system with a “C” shaped core in accordance with embodiments of the present technique;

FIG. 9 illustrates a flow diagram for a method for non-contact powering of a guidewire coil used in accordance with an embodiment of the present technique; and

FIG. 10 illustrates a flow diagram for a method for tracking the guidewire in accordance with an embodiment of the present technique.

### DETAILED DESCRIPTION

For the purpose of illustration only, the following detailed description references a certain embodiment of an electromagnetic tracking system used with an image-guided surgery system. It is understood that the present invention may be used with other imaging systems and other applications.

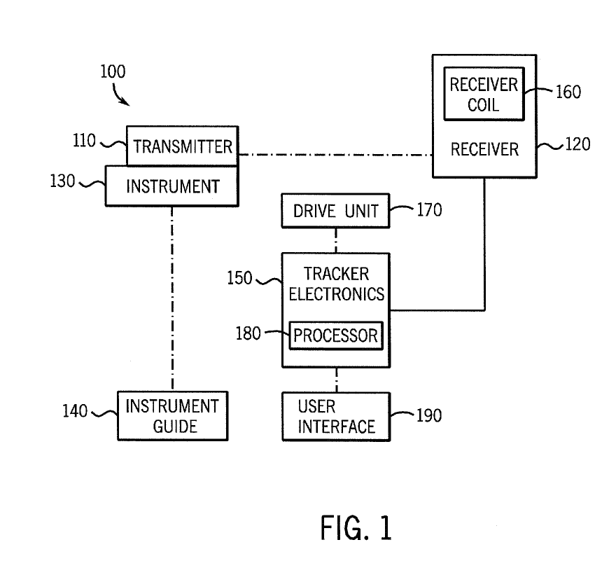

Referring now to FIG. 1, a tracking system 100 in accordance with an embodiment of the present technique is illustrated. The tracking system 100 in certain embodiments generally includes multiple tracking components. As illustrated, the tracking components include a transmitter 110, a receiver assembly 120, an instrument 130, an instrument guide 140, and tracker electronics 150. The receiver assembly including at least one receiver coil 160. In certain embodiments, the system 100 includes a transformer that enables non-contact powering of the instrument 130. In other words, certain embodiments enable the instrument 130, such as a guidewire, to be powered without contact between a power source and the instrument 130.

As discussed in further detail below, embodiments of the system 100 generally include a wired or wireless transmitter 110 that generates a signal (e.g., magnetic field) that is sensed by the receiver assembly 120 and processed via the tracker electronics 150. Processing may then resolve the position and/or orientation of the transmitter 110. In certain embodiments, the transmitter 110 is coupled to the instrument 130 to enable tracking of the instrument 130. The instrument 130 may include a variety of devices, including those used during medical procedures. For example, the instrument 130 may include a guidewire, a drill, a catheter, an endoscope, a laparoscope, a biopsy needle, an ablation device, ultrasound transducers, and flexible ear, nose and throat (ENT) instruments or other medical devices. In an embodiment, the instrument 130 may be tracked with position information relative to the receiver assembly 120. Further, in an embodiment where the relative location of the receiver assembly 120 is known or determined, the position of the instrument 130 may be tracked with respect to the instrument guide 140 or other reference points.

In the tracking system 100, the transmitter 110 may include a conductive coil that generates an electromagnetic (EM) field when a current is driven across the coil. Generally, the EM field may be sensed by a receiver (e.g., the receiver assembly 120) and processed (e.g., processed by the tracker electronics 150) to determine the positional and/or orientation of the transmitter 110. In certain embodiments, the transmitter 110 includes a single dipole coil. The dipole coil generally includes a geometry that may be characterized by various factors. For instance, the geometry of the core of the coil may determine a factor proportional to the effective area (e.g., area factor) of the coil. In one embodiment, the transmitter 110 may include a single dipole coil that is about 8 mm long and about 1.7 mm in diameter, with 7700 turns of American Wire Gauge (AWG) wire formed around a ferromagnetic core that is about 8 mm long and about 0.5 mm in diameter. For example, the area factor is defined by the following equation:

$$area\_factor = {( \frac{coil\_length}{coil\_diameter} )}^{2} . \qquad ( 1 )$$

Thus, the effective coil area factor of the exemplary coil is approximately 22, e.g., (8 mm/1.7 mm)². This factor may be used to characterize a specific coil with respect to other coils. Based on this characterization as well as others, the dipole coil may be tracked in regards to its position, orientation, and gain (or strength).

When a current is provided across a single dipole coil, a single magnetic field may be generated with a magnitude moment vector along its “axis.” Multiple transmitting coils may be used in coordination to generate multiple magnetic fields. For example, the transmitter 110 may include three co-located orthogonal quasi-dipole coils (e.g., a coil-trio). When a coil-trio is energized, each coil generates a magnetic field. As a result, three magnetic fields are generated with magnitude vectors that are co-located and orthogonal to one another. For example, in an embodiment, the transmitter 110 includes an ISCA (Industry Standard Coil Architecture), such as an ISCA coil trio, for example. ISCA is defined as three approximately collocated, approximately orthogonal, and approximately dipole coils.

In a further embodiment, the transmitter 110 may include a wireless configuration. In other words, the transmitter 110 may operate without a wired connection to the receiver 120 and/or the tracker electronics 150. Accordingly, an embodiment including the wireless transmitter 110 eliminates the need for a cable connecting the transmitter 110 and/or the instrument 130 to the tracker electronics 150. In one embodiment, the receiver assembly 120 may be wired to other system components while the transmitter 110 is disposed without a wired connection to other system components 100, such as the tracker electronics 150. In an embodiment, the transmitter 110 may include a single-coil wireless transmitter. An example of a single-coil wireless transmitter may be found at U.S. Pat. No. 7,158,754, entitled “Electromagnetic Tracking System and Method Using a Single-Coil Transmitter,” issued Jan. 2, 2007 and filed on Jan. 6, 2005, with inventor Peter Anderson, which is herein incorporated by reference.

Further, the system 100 may include various characteristics conducive to wireless operation. For example, software running with the tracker electronics 150 may be reconfigured to accommodate a wired or wireless transmitter 110. In other words, the tracker electronics 150 may include processing to determine the output of the transmitter 110, and to process signals sensed by the receiver assembly 120. In an embodiment, the wireless transmitter 110 may include an oscillator or other current source that is independent (e.g., isolated) from the tracker electronics 150 and the receiver assembly 120. In such an embodiment, the oscillator may drive a current across the coil at a desired frequency. Further, the transmitter 110 may receive power from a variety of sources. In one embodiment, the transmitter 110 may be powered via the instrument 130. In an alternative embodiment, the transmitter 110 includes a battery-powered driver and receives a clock signal from the tracker electronics 130 via a magnetic, radio frequency, ultrasonic, or other signal generator. Such a clock signal may eliminate phase-locking and ambiguity in the sign of the gain of the transmitter 110.

In another embodiment, the transmitter 110 may receive power from a separate power source. For example, the separate power source may include a battery, photocells powered by ambient light, or rectified radio frequency energy rectified. In an embodiment including a photocell, a small silicon photocell is connected across a coil of the transmitter 110. The photocell is illuminated with amplitude-modulated light to power a driver for the transmitter 110. Alternatively, two photocells may be connected in antiparallel across the coil of the transmitter 110 and by alternately illuminating each photocell, an alternating current may be generated in the coil. Alternate illuminations may be achieved using two optical fibers (one to each photocell) or using one fiber to illuminate the photocells through filters of different polarizations or different colors, for example. In another embodiment, two photocells sensitive to different wavelengths of light may be integrated on top of each other. The optically powered coil may enable reduced wire sizes and may also reduce the amount of electrical energy that may otherwise be dissipated by electrical wiring.

As discussed previously, the transmitter 110 may be coupled to the instrument 130 to enable tracking of the instrument 130. For example, in an embodiment, the transmitter 110 is integrated within the instrument 130. In other words, the transmitter 110 may be built-in and/or internal to the instrument 130, as opposed to being coupled to the exterior of the instrument 130. For example, the transmitter 110 may be integrated into the tip of a guidewire to enable tracking of the guidewire. In another embodiment, a single transmitter coil may be located at the tip of a catheter, for instance.

The system 100 may also include one or more additional transmitters 110 for use in tracking the instrument 130. These additional transmitter(s) may be wired or wireless transmitter(s). For example, a second transmitter may be located on the instrument guide 140 or on the instrument 130 and wired to the tracker electronics 150. The transmitter 110 and additional transmitter(s) may be tracked simultaneously from the receivers in the receiver assembly 120.

Further, the magnetic field emitted by the transmitter 110 may enable measurement of position, as well as communication with the receiver assembly 120 and the tracker electronics 150. For, example a communication signal may be embedded in the transmitted EM field, and the signal processed to interpret the embedded information. In other words, the signal (e.g., the current) that generates the magnetic field may include multiple frequencies that can be resolved to determine position and other information. For example, in one embodiment, the wireless transmitter 110 may be combined with various wireless radio frequency identification (RFID) schemes that enable identification and/or data transfer without a direct electrical (e.g., wired) contact between the transmitter 110 and the receiver assembly 120 or tracker electronics 150.

Complementary to the transmitter 110, the system 100 may also include at least one receiver assembly 120 that is configured to “receive” (e.g., sense) the magnetic field(s) generated by the transmitter 110. The receiver assembly 120 may include at least one receiver coil. Accordingly the receiver assembly 120 may include a single receiver coil or multiple receiver coils. For example, in the illustrated embodiment, the receiver assembly 120 includes at least one receiver coil 160. Accordingly, when a current is applied to the transmitter 110, the magnetic field generated by the transmitter 110 may induce a voltage into the at least one receiver coil 160. As will be appreciated, the induced voltage may be indicative of the mutual inductance between the transmitter 110 and the at least one receive coil 160. Thus, the induced voltage across the at least one receiver coil 160 may be sensed and processed to determine the mutual inductance (Lm) between the transmitter 110 and each of the at least one receiver coil 160.

Similar to the transmitter 110, the at least one receiver coil 160 may employ a single dipole coil or multiple coils (e.g., a coil trio). For example, the system 100 may include an electromagnetic tracking system configured with industry-standard coil architecture (ISCA). ISCA type coils defined as three approximately collocated, approximately orthogonal, and approximately dipole coils. An ISCA configuration includes a three-axis dipole coil transmitter 110 and a three-axis dipole coil at least one receiver 160. In such a configuration, the coils of the transmitter 110 and the coils of the at least one receiver coil 160 are configured such that the three coils exhibit the same effective area, are oriented orthogonally one another, and are centered at the same point. Using this configuration, nine parameter measurements may be obtained (e.g., a measurement between each coil of the transmitter and each receiving coil). From the nine parameter measurements, processing may determine position and orientation information for each coil of the transmitter 110 with respect to each coil of the at least one receiver coil 160. If either of the transmitter 110 or the at least one receiver coil 160 is in a known position, processing may also resolve position and orientation relative to the known position.

The receiver assembly 120 may include a variety of receiver combinations and configurations. For example, in one embodiment, the receiver assembly 120 may include a plurality of at least one receiver coils 160 disposed on a circuit board. In another embodiment, the at least one receiver coil 160 may include a sensor coil or telecoil, such as a telecoil coil used in a hearing aid to pick up magnetic audio signals. In an additional embodiment, the at least one receiver coil 160 may include a twelve-coil wired receiver. In another embodiment, the at least one receiver coil 160 may include an array(s) of three-axis dipole wire-wound coil trios. Due to inaccuracies in coil winding, the at least one receiver coils 160 may be characterized before use in tracking. Further, the wire-wound receiver coil arrangement may have a better signal-to-noise ratio than a printed circuit board coil, due to a larger volume of copper in a wound coil of a given volume. Additionally, POG (position-orientation-gain) seed algorithms may be used with characterized receiver coils, for example.

In the illustrated embodiment, the receiver assembly 120 is not directly coupled to the instrument guide 140. However, in another embodiment, the receiver assembly 120 may be attached to the instrument guide 140. Attaching the receiver assembly 120 to the instrument guide 140 may provide a reference for tracking the transmitter 110 relative to the instrument guide 140. In an embodiment, the instrument guide 140 may include a drill guide or other medical instrument guide, for example. In another embodiment, the instrument 130 with instrument guide 140 may be a tool that is indirectly controlled for applications wherein an operator's field of vision is obscured by an object. For example, in an embodiment, the instrument 130 and instrument guide 140 may include a guidewire coupled to a drill guide that directs the guidewire into a cavity (e.g., vein) of a patient.

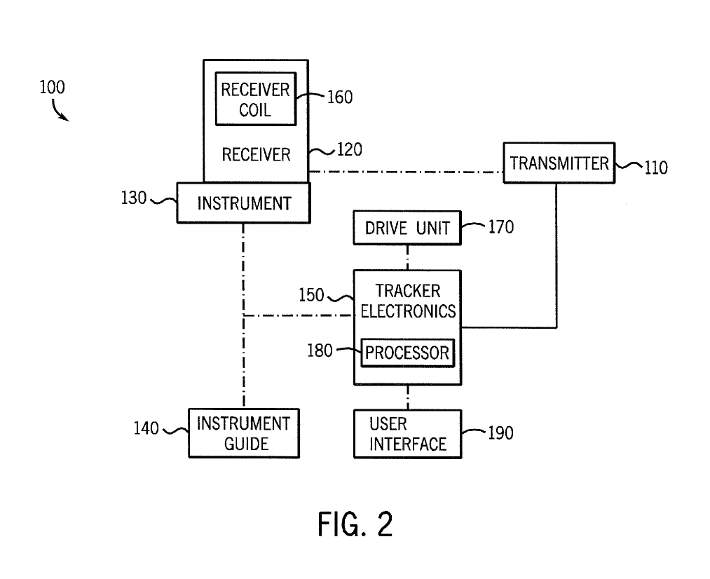

As will be appreciated, the mutual inductance of the transmitter 110 and the at least one receiver coil 160 is the same, regardless as to which sensor generates the electromagnetic field. Therefore, positioning and functionality of the transmitter 110 with respect to the receiver 160 may be reversed. For example, as illustrated in FIG. 2, the receiver assembly 120 may be coupled to the instrument 130, and the transmitter 110 located external to the instrument 130. Accordingly, the magnetic field may be generated from the transmitter 110 external to the instrument, and received by the receiver assembly 120 and receiver coil 160 disposed internal to or proximate to the instrument 130 (e.g., a guidewire or catheter).

Referring again to FIG. 1, the system 100 may further include a drive unit 170. In accordance with certain implementations of the present technique, the drive unit 170 may be configured to provide a drive current to the coil of the transmitter 110. By way of example, a drive current may be supplied by the drive unit 170 to energize a coil of a transmitter 110 and, thereby, generate a magnetic field that is sensed by the receiver assembly 120. The drive current may include a periodic waveform with a given frequency (e.g., a sine wave). In turn, the current across the coil of the transmitter 110 will generate a magnetic field at the same frequency as the drive current. For example, electromagnetic tracking systems generally may be supplied with sine wave current waveforms with frequencies between 8 kHz and 40 kHz and, thus, generate magnetic fields with frequencies between 8 kHz and 40 kHz. In an embodiment, the coil of the transmitter 110 is driven with a continuous wave (CW) sine wave (a 20 kHz sine wave, for example). In one embodiment, the drive unit 170 may be integral to the tracker electronics 150. For example, in one embodiment, the drive unit 170 may be powered via a connection to the AC power grid. While the drive unit 170 and the tracker electronics 150 may be a single unit, as illustrated, in certain embodiment, the drive unit 170 may be provided as a separate unit. For example, the drive unit 170 may be integral to the instrument 130 or the transmitter 110. For example, in an embodiment, such as a wireless transmitter 110, the drive unit 170 may be powered by an integral 3-volt lithium cell or one of the above discussed techniques, for example. Further, in certain embodiments, the drive unit 170 may be connected to the coil of the transmitter 110 using a short cable (such as a 0.1 meter coaxial cable).

As discussed previously, the tracker electronics 150 may provide for processing of received signals, and may also provide other control and processing functions of the system 100. In one embodiment, the tracker electronics 150 may include a processor 180. The processor 180 may include, for example, a digital signal processor, a CPU, or the like. In the illustrated embodiment, the magnetic fields sensed by the receiver assembly 120 may be output to the processor 180 for processing. Accordingly, the processor 180 may process the received signals to track the position and orientation of the instrument 130. For example, the at least one receiver coil 160 may produce output signals that are indicative of the mutual inductance between a transmitter 110 and the at least one receiver coil 160. Thus, processing may compare the mutual inductances of the received signals to resolve the position of the instrument 130.

When the fields generated include multiple frequencies, processing may be able to determine the magnetic field frequency from the signal sensed by the at least one receiver coil 160. Thus, the frequency of a magnetic field may be useful to distinguish multiple magnetic fields that are sensed by a single receiving coil. For example, when driving a transmitter (such as transmitter 110) having a single dipole coil, a single drive current of a given frequency may be sufficient to identify the magnetic field. This is true because only a single transmitting coil is generating a magnetic field. However, when a transmitter (such as transmitter 110) includes multiple coils (e.g., a coil trio), each of the at least one receiver coils 160 may sense multiple magnetic fields simultaneously. The result may be a single signal from each of the at least one receiver coils 160 that is transmitted to the processor 180. To enable subsequent processing to readily identify each of the magnetic fields, the frequency of each of the generated magnetic fields may be varied. By identifying each magnetic field, processing may be able to isolate the signal between each respective transmitting coil and at least one receiving coil 160 and, thereby, determine the relative position and/or orientation of each of the coils. For example, if each coil of the transmitter 110 is provided a current waveform of a different frequency, processing may identify each magnetic field. Thus, the processor 180 and the tracker electronics 150 may implement any suitable algorithm(s) to establish the position and orientation of the transmitter 110 relative to the receiver 160. For example, the processor 180 may use the ratios of mutual inductance between each coil of the at least one receiver coil 160 and each coil of the transmitter 110 to triangulate the position of the coils. The processor 180 may use these relative positions to resolve a position and orientation of the transmitter 110.

For example, in one embodiment, the processor 180 may use an iterative approach to arrive at a determined position and orientation of the transmitter 110. For example, an initial “seed” approximation of position and orientation may be provided, or resolved by initial measurements of the system 100 and the processor 180. The processor 180 may then use this approximate position and orientation in subsequent algorithms to predict the electric field characteristics and to determine a new estimate of position. The processor 180 may then consider calculating new estimates of the magnetic field characteristics. The iteration of estimating and comparing may continue until the estimated values are sufficiently similar to the position and orientation actually sensed.

Methods and techniques for processing the sensed signals to determine the relative position and orientation of the transmitter 110 and receivers may be found at U.S. Pat. No. 7,158,754, entitled “Electromagnetic Tracking System and Method Using a Single-Coil Transmitter,” issued Jan. 2, 2007 and filed on Jan. 6, 2005, with inventor Peter Anderson, which is herein incorporated by reference.

As illustrated, the system 100 may also include a user interface 190 integrated into the tracker electronics 150. For example, the user interface 190 may include a monitor to display the determined position and orientation of a tracked object. As will be appreciated, the user interface 190 may include additional devices to facilitate the exchange of data between the system 100 and the user. For example, the user interface 190 may include a keyboard, mouse, printers or other peripherals. While the tracker electronics 150 and the user interface 190 may be a single unit, as illustrated, in certain embodiments, the user interface 190 may be provided as a separate unit.

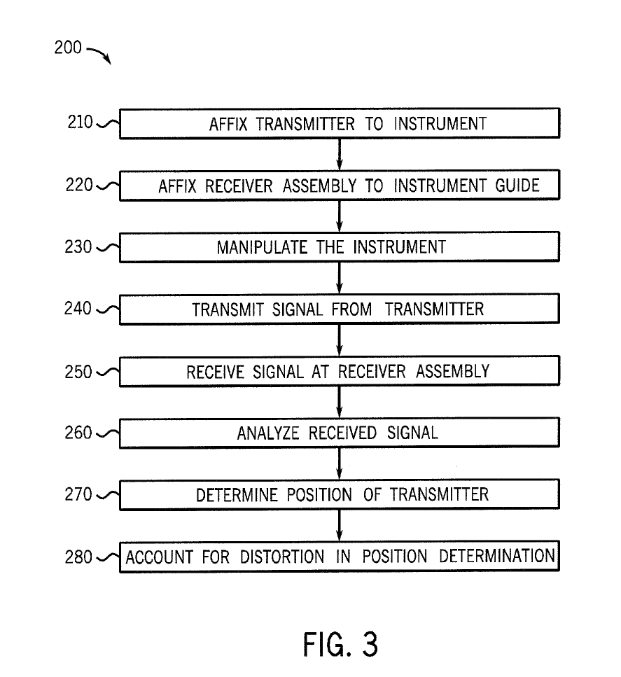

FIG. 3 illustrates a flow diagram for a method 200 for tracking a position of an instrument 130 in accordance with an embodiment of the present technique. The method 200 includes affixing the transmitter to an instrument, as illustrated at block 210. For example, in one embodiment, the transmitter 110 is affixed to the instrument 130, such as a catheter, guidewire, or other medical instrument or tool.

The method 200 also includes affixing the receiver assembly to the instrument guide, as illustrated at block 220. For example, in one embodiment, the receiver assembly 120 may be affixed to an instrument guide 140. Other embodiments may include affixing the receiver 120 to other locations relative to the transmitter 110, such as a patient or a table proximate the patient. The step of affixing the receiver assembly may be performed before or after affixing the transmitter to the instrument.

The method 200, next, includes manipulating the instrument, as illustrated at block 230. For example, an operator may manipulate the instrument 130 inside the patient using the instrument guide 140 (e.g., a surgical drill). Further, the method 200 includes transmitting a signal from the transmitter, as illustrated at block 240. For example, the transmitter 110 may be energized to broadcasts a signal using power from the drive unit 170 or other source. In other words, a current may be passed across a coil of the transmitter 110 to generate an electromagnetic field.

Next, the method 200 includes receiving a signal at a receiver assembly, as illustrated at block 250. For example, in one embodiment, the at least one receiver coil 160 of the receiver assembly 120 may sense the signal transmitted from the transmitter 110 (block 240). In other words, the at least one receiver coil 160 may have a voltage or current induced across it due to the mutual inductance between the coils of the transmitter 110 and the coils of the at least one receiver coil 160. Following receiving the signal, the method 200 includes analyzing the received signal, as illustrated at block 260. For example, in an embodiment, the tracker electronics 150 and the processor 180 measure the signals received by the at least one receiver coil 160. Analyzing may include measurements relating to mutual inductance, filtering, and the like.

Further, as illustrated at block 270, the method 200 includes determining the position of a transmitter. As discussed previously, determining the position of the transmitter 110 may include further processing of the received and transmitted signals to determine the position of the transmitter 110 with respect to the receiver assembly 120, the instrument guide 140 or other reference coordinate system, for example. Further, the direction or orientation of the transmitter 110 may be determined from the received signals. Processing may include the use of triangulation to determine the position of the transmitter 110 relative to the positional relationship between the at least one receiver coil 160 in the receiver assembly 120, for example. In an alternative embodiment, multiple transmitters 110 transmit signals to the receiver assembly 120 to provide additional signal inputs to aid processing in resolving the position of the instrument 130. Further, as illustrated at block 280, the method 200 includes accounting for distortion in position determination. For example, integral (e.g., Green's function) or differential (e.g., finite-element) methods may be used to determine an impact of field effects from a distorter on the tracked position of the transmitter 110.

As will be appreciated, tracking systems 100, such as those discussed previously may be used in a variety of applications. For example, the tracking system 100 may be employed to provide for tracking of a guidewire or other instrument internal to a patient. Thus, the location of a guidewire that is disposed internal to a patient may be tracked to ensure that a catheter slid over the guidewire is inserted in the correct location internal to the patient. The embodiments discussed below include a guidewire system 300 and method that enables non-contact powering of the transmitter 110 in the instrument 130 and enables the location of the instrument 130, such as a guidewire, to be continuously tracked as another medical device, such as a catheter, is positioned relative to (e.g., slid over) the instrument 130.

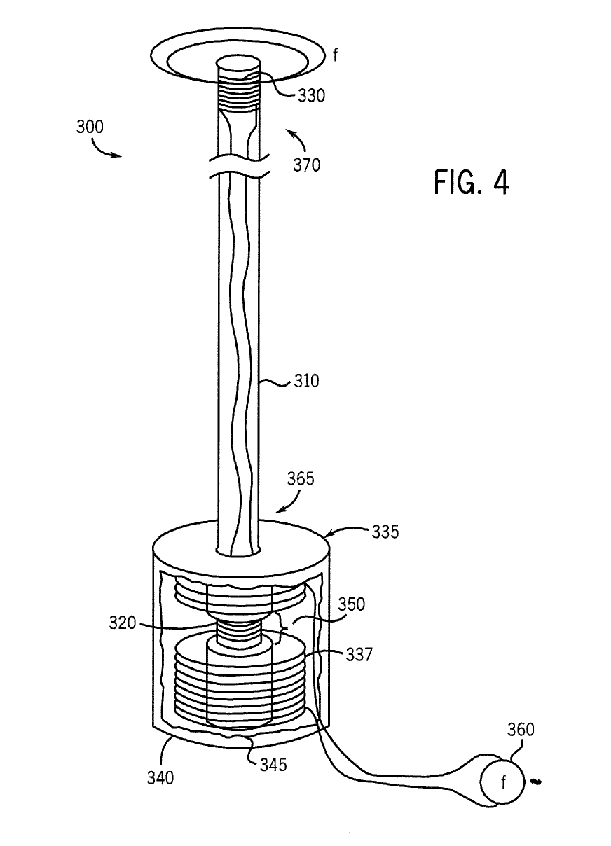

FIG. 4 illustrates a guidewire system 300 that includes a transformer coupling in accordance with an embodiment of the present technique. As illustrated, the guidewire system 300 includes a guidewire 310, a pickup coil 320, a transmitter coil 330, and a transformer 335. The transformer 335 includes a primary winding 337, a core 340, a central axis hole 345, and an air gap 350. Further, the system 300 includes a frequency source 360.

The guidewire 310 may include an instrument used for positioning or guiding other medical devices. For example, in certain embodiments, the guidewire 310 includes a long cylindrical body that may be threaded into a vein or other cavity of a patient to provide a path for the insertion of other medical devices (e.g., a catheter). In the illustrated embodiment, the guidewire 310 includes a proximal end 365 and a distal end 370. During use, the distal end 370 is generally inserted into a patient, and the proximal end 365 is generally used to position, guide, or otherwise control the distal end 370 of the guidewire 310.

In the illustrated embodiment, the pickup coil 320 is embedded or otherwise positioned in the proximal end 365 of the guidewire 310. The pickup coil 320 may include a single dipole coil, a coil trio, or the like, as discussed previously. Accordingly, disposing the pickup coil 320 in a magnetic field may induce a voltage/current across the pickup coil 320.

The transmitter coil 330 may be embedded or otherwise coupled to the distal end 370 of the guidewire 310. The transmitter coil 330 may include a single dipole coil, a coil trio, or the like, as discussed previously. Accordingly, providing a current across the transmitter coil 330 may produce at least one electromagnetic field that is sensed (e.g., sensed by the receiver assembly 120). In an embodiment, the transmitter coil 330 may be similar or identical to the pickup coil 320.

The transformer 335 includes components to enable inducing a current or voltage into at least one pick up coil 320. For example, in the illustrated embodiment, the transformer 335 includes the primary winding 337 wrapped around a core 340. In an embodiment, the core 340 includes a ferrite pot-core assembly, for example. The illustrated pot-core assembly includes a bobbin, post, or pin. In an embodiment, the winding 337 includes a ferrite material surrounding the outside of a central rod of the core 340. For example, in the illustrated embodiment, the primary winding 337 is wound around the bobbin, post or pin. Further, in an embodiment, the primary winding 337 may be insulated copper windings or other electrically-conductive wire windings. Further, the “air-gap” 350 or region of close-to-unity magnetic permeability may be positioned around the central post in the core 340. The air-gap 350 enables the magnetic flux generated by the primary winding 337 to induce a current in the pickup coil 320 located in the air-gap 350. For example, to form the air-gap 350, flux may be added into the air in the center of the primary winding 337 that surrounds the guidewire 310. In an embodiment, the air-gap 350 may be filled with a non-magnetic electrically-insulating ceramic, for example. Further, the length of the air-gap 350 may be enlarged to be longer than the length of the pickup coil 320.

The center of the transformer 335 may include a hole that enables the guidewire 310 and the pickup coil 320 to be disposed internal to the primary winding 337. For example, the illustrated embodiment includes a central axial hole 345 that is large enough in size (e.g., diameter) to pass the guidewire 310 through at least a portions of the central axis hole 345. In one embodiment, the central axis hole 345 extends completely through the length of the transformer such that at least a portion of the guidewire 310 can pass entirely through the transformer 335. In other words, the central axis hole 345 enables the transformer 335 to be positioned at locations along the length of the guidewire 310, in addition to the proximal end 365.

Further, the transformer 335 may include a single unit that is isolated, insulated, or generally sealed from the external environment. For example, in an embodiment, insulation, such as high-temperature electrical insulation, may be provided about the exterior of the transformer 335. High-temperature electrical insulation on magnet wire and ceramics, for example, may withstand temperatures of autoclaving to maintain a hygienic environment. Thus, the entire transformer 335 may be made autoclavable or otherwise sterilizable. For example, the addition of a moisture resistant insulation layer about the transformer 335 may enable the transformer to be exposed to fluids used during the sterilization process. Similarly, the guidewire 310 may include a single unit that is insulated. For example, in one embodiment, the guidewire 310 includes an insulation layer that generally seals the exterior of the guidewire 310 electrically and thermally, and that enables autoclaving of the guidewire 310.

In an embodiment, providing a current across the primary winding 337 may induce a current into a complementary coil. For example, in an embodiment where the pickup coil 320 is disposed internal to the primary winding 337, providing a current across the primary winding 337 at a given frequency may induce a current/voltage across the pickup coil 320. Accordingly, in an embodiment with the pickup coil 320 electrically coupled to the transmitter coil 330, power (e.g., inductive power) may be provided to the primary winding 337 to provide power to transmitter coil 330. In other words, the pickup coil 320 is inductively coupled to the winding 337, such that driving a current across the primary winding 337 induces a current across the pickup coil 320 and the current drives the same or similar current across the transmitter coil 330. Thus, an electrical connection to drive the transmitter coil 330 is replaced with magnetic coupling, for example.

As discussed previously, it may be desirable to drive the transmitter coil 330 at a given frequency that is beneficial to tracking. Thus, the primary winding 337 may be powered at a desired transmitter frequency, for example. In certain embodiments, the winding 337 and, thus, the pickup coil 320 and the transmitter coil 330, are powered at any frequency (f) from a frequency source 360. For example, the frequency source 360 may provide a current with a given frequency (f). In certain embodiments, the primary winding 337 and, thus, the pickup coil 320 and the transmitter coil 330 are powered at a frequency ‘f’ between 25 Hz and 33 kHz, for example. Accordingly, power with a desired frequency is provided to the pickup coil 320 and the transmitter coil 330 of the guidewire 310 without a direct electrical contact. In other words, power is transmitted to the guidewire 310 without direct wiring. Thus, the guidewire 310 may be formed, without creating bulges in the guidewire 310 that would typically be used for electrical connections. Accordingly, due to the integral nature of the guidewire 310 and pickup coil 320 and transmitter coil 330, the guidewire 310 can be autoclaved or otherwise sterilized without affecting the power-generating coils, for example. Further, the lack of external electrical connections may be conducive to safety concerns.

The guidewire 310 may also include a plurality of transmitter coils 330. Each of these transmitter coils 330 may be driven by a common or different power sources with the same or different frequencies. In one embodiment of the guidewire 310 including a plurality of transmitters 330 at or near its distal end 370, a number of transformers 335 and/or primary windings 337 corresponding to the number of transmitters 330 may be stacked. In such an embodiment, the guidewire 310 may include a plurality of pickup coils 320 that are each coupled to one of the plurality of transmitter coils 330. In other words, each pickup coil 320 may be aligned to a corresponding primary winding 337. Accordingly, each transformer 335 may be energized to drive a separate pickup coil 320 to power each of the transmitter coils 330, for example. Alternatively, multiple pickup coils 320 may be powered using a single transformer 335 or primary winding 337. For example, a single transformer 335 or primary winding 337 may be inductively coupled to multiple pickup coils 320 simultaneously, such that energizing the primary winding 337 of the transformer 335 induces a current into each pickup coil 320 and, thus, simultaneously powers the transmitter coils 330.

In another embodiment, a plurality of tracking systems may be located on the guidewire 310 and other medical devices. For example separate transformer systems may be used to track the guidewire 310, and a similar system 300 employed on a catheter to track the catheter.

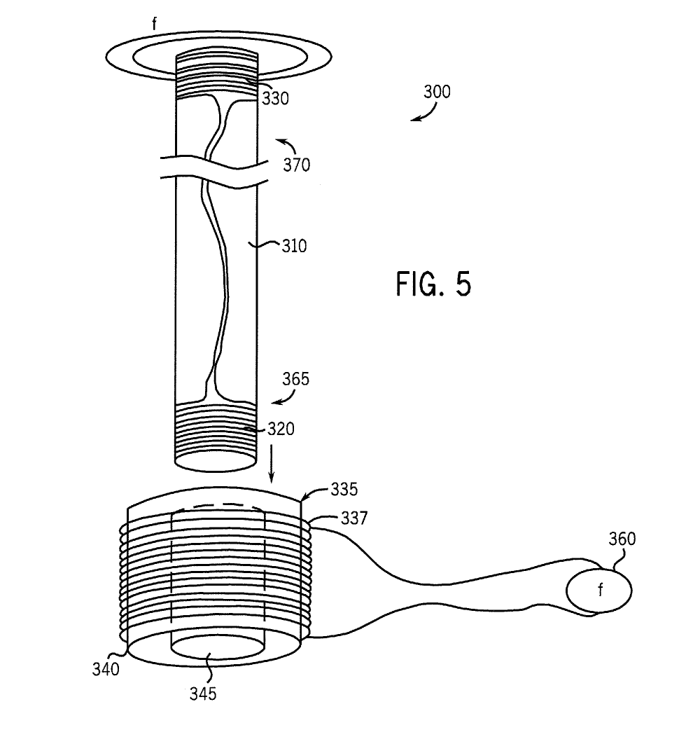

FIG. 5 illustrates the guidewire system 300 with the transformer 335 including a solenoidal coil in accordance with another embodiment of the present technique. In the illustrated guidewire system 300, the primary winding 337 includes a solenoidal coil that is wound to have a length longer than the pickup coil 320. Further, the transformer 335 includes the central axis hole 345 with a bore having a diameter large enough to pass at least the portion of the guidewire 310 including the pickup coil 320 into the central axis hole 345. Accordingly, when the guidewire 310 and the pickup coil 320 is disposed in the center of the primary winding 337, power provided across the solenoidal coil of the primary winding 337 induces a current into the pickup coil 320 and the transmitter coil 330. Thus, the inductive coupling between the primary winding 337 and the pickup coil 320 may be employed to power the transmitter coil 330.

As discussed above, the guidewire system 300 may be used during the disposal of the guidewire 310 internal to the patient. For example, the position of the guidewire 310 (e.g., the position of the distal end 330) may be tracked to insure the guidewire 310 is properly located in a vein or other cavity of the patient. In other words, during insertion of the guidewire 310, the transmitter coil 330 may be energized to generate a magnetic field that is sensed by the receiver assembly 120 and processed by the tracker electronics 150 to resolve the position and/or orientation of the guidewire 310. Subsequent to disposing the guidewire 310, other medical devices are generally disposed in the patient using the guidewire 310 as a positional reference. For example, in medical applications that include disposing a catheter (e.g., a hollow tube) in the patient, the catheter is threaded over the guidewire 310, the catheter slid up the guidewire 310 into position, and the guidewire removed. Accordingly, in an embodiment where the central axis hole 345 is only opened at one end (e.g., not a through hole), the guidewire 310 may be removed from the transformer 335 prior to the catheter 310 being slid over the guidewire 310. Thus, during the time the guidewire 310 is removed from the central axis hole 345 of the transformer 335, the system 300 may not be able to track the position and/or orientation of the guidewire 310 because the pickup coil 320 is no longer inductively coupled to the primary winding 337 of the transformer 335.

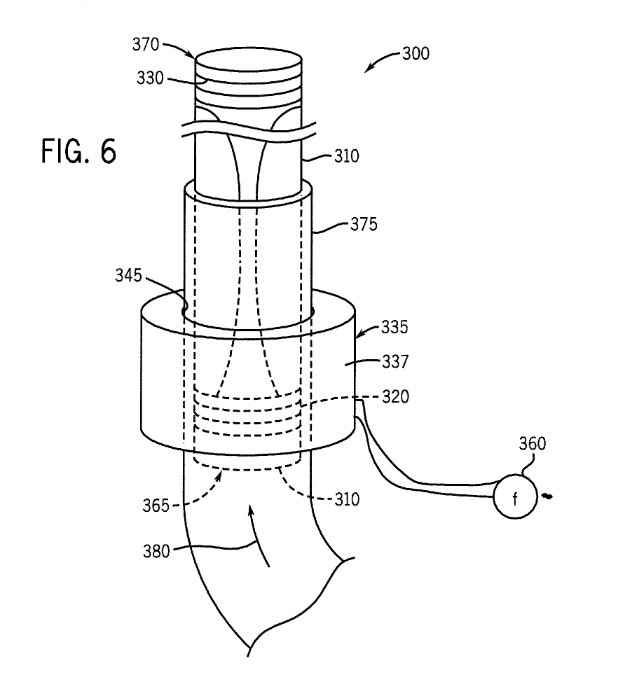

FIG. 6 illustrates an embodiment of the guidewire system 300 that enables the a catheter 375 to be disposed over the guidewire 310 while the pickup coil 320 remains inductively coupled to the primary winding 337 of the transformer 335. For example, in the illustrated embodiment, the central axis hole 345 is a through hole that includes a diameter that is large enough to accept the proximal end 365 of the guidewire 310 and the catheter 375. For example, in the illustrated embodiment, the catheter 375 includes a hollow tube that is slid in the direction of arrow 380, over the proximal end 365 of the guidewire 310 and through the central axis hole 345. Generally, the catheter 375 may be further slid/threaded onto the guidewire 310 until the catheter is disposed near the distal end 370 of the guidewire 310, or any suitable position. Accordingly, the position of the guidewire 310 may continue to be tracked as the catheter 375 is slid into position. In other words, because the guidewire 310 remains in the central axis hole 345, the pickup coil 320 may continue to be inductively coupled to the primary winding 337 of the transformer 335, enabling powering of the transmitter coil 330 during the placement of the catheter 375.

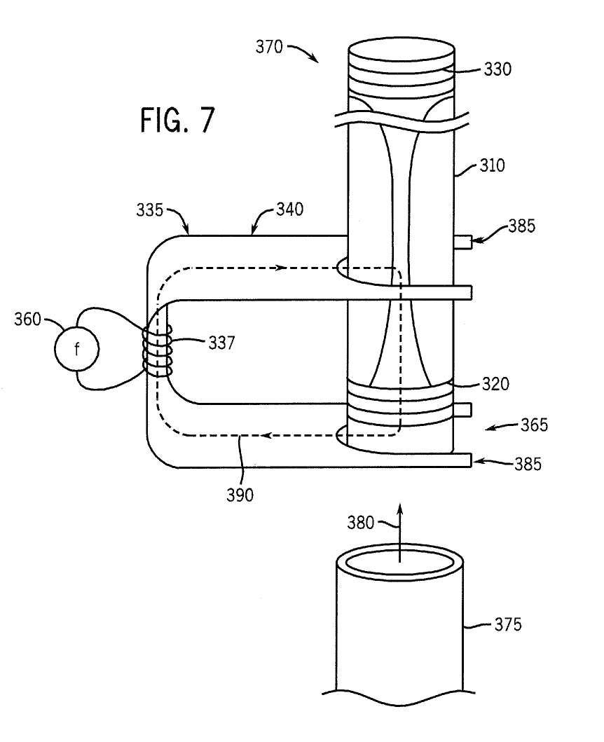

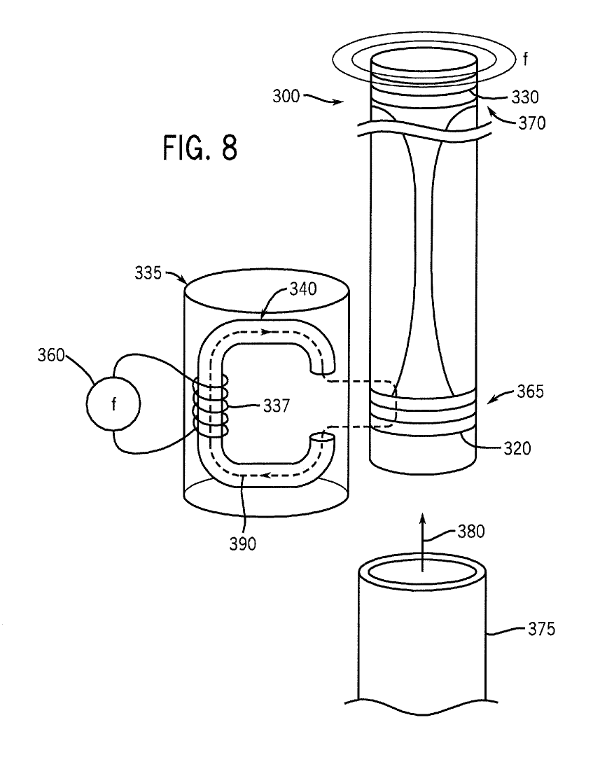

FIG. 7 illustrates an alternate embodiment of the guidewire system 300 wherein the transformer 335 enables powering the transmitter coil 330 without passing the guidewire 310 into a hole of the transformer 335. In certain embodiments, the core 340 is configured to transmit a magnetic flux for powering a complementary coil. In the illustrated embodiment, the core 340 of the transformer 335 includes a C-shaped body having the primary winding 337 disposed about a portion of the core 340. Accordingly, providing a current across the primary winding 337 induce a magnetic flux into the core 340 in the direction of arrow 390. Further, the embodiment illustrated in FIG. 7 includes slotted ends 385. The slotted ends 385 include recesses in the core 340 that generally conform to the shape of the guidewire 310 to aid in focusing the magnetic field to the guidewire 310. The focused magnetic field may provide for increased magnetic coupling to the wire and coils 320 and 330 internal to the guidewire 310. Thus, the magnetic field generated as a result of the magnetic flux, passes through the pickup coil 320 of the guidewire 310, thereby, inducing a current into the pickup coil 320 and the transmitter coil 330. Accordingly, energizing the primary winding 337 with the frequency source 360 may drive a current with a given frequency across the transmitter coil 330, generating a magnetic field that is sensed and processed by the receiver assembly 120 and the tracker electronics 150. In an alternate embodiment, the ends of the C-shaped coil may terminate proximate the exterior of the guidewire 310. For example, FIG. 8 illustrates an alternate embodiment of the C-shaped coil in the transformer 335. The “C” shaped core 340 may be disposed proximate to the pickup coil 320 to provide a magnetic flux that induces a current into the pickup coil 320, as discussed above. In either of the embodiments of FIGS. 7 and 8, the transformer 335 may include a housing conducive to the placement of the core 340 proximate the guidewire 310. For example, in certain embodiments the transformer 335 may include an indentation to aid in the placement of the guidewire 310 proximate the core 340. In one embodiment, the indentations may conform to the slots 385 to ensure a practitioner can readily align the guidewire 310 and the catheter 375 to the core 340.

In an embodiment, the pickup coil 320 of the guidewire 310 may be powered by positioning the guidewire 310 proximate to the transformer 335. For example, the exterior body of the transformer 335 may include an indentation (e.g., slots 385), similar to those discussed above, that is conducive to accepting and positioning the guidewire 310. Accordingly, the guidewire 310 and the pickup coil 320 may be moved or secured proximate to the transformer 335 and the core 340 such that the magnetic field generated by energizing the primary winding 337 induces a current into the pickup coil 320 and powers the transmitter coil 330. As will be appreciated, such a configuration may also simplify disposing the catheter 375 over the guidewire 310. For example, the catheter 375 may be slid in the direction of arrow 380, and over the guidewire 310 while the guidewire 310 remains proximate the transformer 335. Accordingly, the transmitter coil 330 may continue to be powered, and the guidewire 310 tracked as the catheter is slid over the guidewire 310. In such an embodiment, the diameter of the catheter 375 may be varied without significant issues of clearance between the guidewire 310 and the transformer 335.

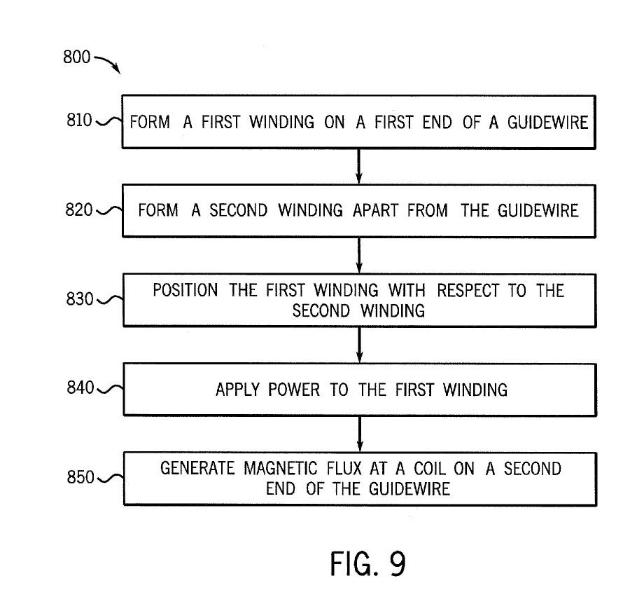

FIG. 9 illustrates a flow diagram for a method 800 for non-contact powering of a guidewire 310 in accordance with an embodiment of the present technique. As illustrated at block 810, the first step of the method 800 includes forming a first winding on a first end of a guidewire. In one embodiment, forming a first winding may include disposing the pickup coil 320 integral to the proximal end 365 of the guidewire 310. Accordingly, an embodiment may also include positioning the pickup coil 320 such that it can be inductively coupled to a complementary coil (e.g., the primary winding 337).

The method 800 includes forming a second winding apart from the guidewire, as illustrated at block 820. In an embodiment, forming the second winding may include assembly the primary winding 337 to the core 340 of the transformer 355, for example. As discussed previously, the primary winding 337 may be disposed about a pot-core, a “C” shaped core, and the like. Accordingly, the primary winding 337 may be energized to induce a current in the core 340, or a complementary coil (e.g., the pickup coil 320).

Next, the method 800 includes positioning the first winding with respect to the second winding, as illustrated at block 830. In one embodiment, the proximal end 365 of the guidewire 310 is positioned inside the core 340 of the transformer 355 such that the primary winding 337 forms the primary winding of a non-contact transformer, and the pickup coil 320 forms the secondary windings of a non-contact power transformer that provides power to the guidewire 310 and the transmitter coil 330. In another embodiment, positioning the first winding with respect to the second winding (block 830) may include merely positioning the guidewire 310 and pickup coil 320 proximate the exterior of the transformer 355. For example, in an embodiment where the core 340 of the transformer 355 includes a “C” configuration, the guidewire 310 and pickup coil 320 may be placed against the exterior of the transformer 355 to enable inductive coupling to power the transmitter coil 330. Further, in an embodiment where the core 340 includes an indentation (e.g., slots 385), the guidewire 310 may be disposed in the indentation to properly align the pickup coil 320 to the core 340 and the primary winding 337.

Further, the method 800 includes providing power to the first winding, as illustrated at block 840. In one embodiment, providing power to the first winding (block 840) includes inductively coupling the first winding to a complementary winding. For example, in an embodiment the primary winding 337 (e.g., the second winding) may be energized to create a magnetic field that induces a current into the pickup coil 320 (e.g., the first winding). As discussed previously, the current induced into the pickup coil 320 may be transmitted to power the transmitter coil 330. For example, block 850 illustrates generating magnetic flux at a coil on a second end of the guidewire. For example, the transmitter coil 330 at the distal end 370 of the guidewire 310 may be powered via the power inductively applied to the pickup coil 320 at the proximal end 365 of the guidewire 310. Accordingly, the signal (e.g., the electromagnetic field) generated by the transmitter coil 330 may be used to track the guidewire 310 and/or catheter, for example.

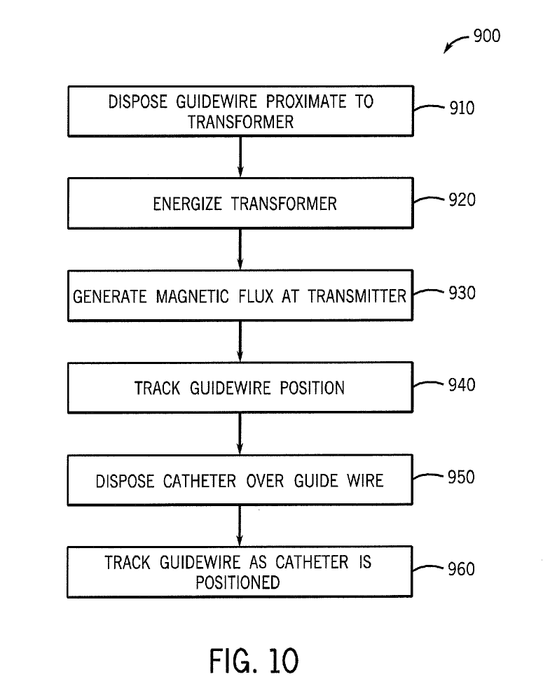

FIG. 10 illustrates a flow diagram for a method 900 of inserting the catheter 375, or other medical instrument over the guidewire 310 while continuing to track the location of the guidewire 310. For example, the first step of the method 900 includes disposing the guidewire proximate to the transformer, as illustrated at block 910. In an embodiment including the transformer 335 having the central axis hole 345 extending completely through the transformer 335, this may include positioning the guidewire 310 into the central axis hole 345 such that the pickup coil 320 is proximate the primary winding 337. In an embodiment wherein the transformer 335 includes the core 340 having a C-shaped configuration, the step (block 910) may include disposing the guidewire 310 and the pickup coil 320 proximate the body of the transformer 335 or the indentation (e.g., slots 385) of the transformer 335 or the core 340.

Next, the method 900 includes energizing the transformer, as illustrated at block 920. In other words, a current may be induced across the primary winding 337 to induce a current in the pickup coil 320. Thus, in an embodiment including the transmitter coil 330 coupled to the pickup coil 320, the method 900 may also include generating magnetic flux at the transmitter coil 330, as illustrated at block 930.

With the transmitter coil 330 generating a magnetic flux (block 930), the method next includes tracking the guidewire position, as illustrated at block 940. For example, in an embodiment, the magnetic flux generated by the transmitter coil 330 is sensed by the receiver assembly 120, and a corresponding signal transmitted to the tracker electronics 150 for processing. This may be accomplished as the guidewire 310 is disposed (e.g., threaded) into the patient. As discussed previously, the tracker electronics 150 may employ various algorithms, filters, and the like to determine the position and/or orientation of the transmitter coil 330, and, thus, the position and/or orientation of the distal end 370 of the guidewire 310.

Next, the method 900 includes disposing the catheter over the guidewire and continuing to track the guidewire as the catheter is positioned, as illustrated at blocks 950 and 960. In other words, in an embodiment, the catheter 375 is slid over the guidewire with the pickup coil 320 and the guidewire 310 remaining in a position that enables the pickup coil 320 to remain inductively coupled to the transformer 335. Thus, while the catheter 375 is inserted into the patient, the transmitter coil 330 continues to generate a signal, and the system 100 continues to track the location of the guidewire 310. This may be particularly useful to ensure that the guidewire 310 is not inadvertently moved relative to the patient or a target location while the catheter 375 is being inserted. For example, if the guidewire 310 and the pickup coil 320 are not inductively coupled to the transformer 335 during insertion of the catheter 375, the transmitter coil 330 is not powered and, thus, positioning of the guidewire 330 and the catheter 375 may be confirmed by having to reenergize the pickup coil 230 after the catheter 375 is disposed in the patient. In other words, the guidewire 310 is removed from the transformer 335 during insertion of the catheter 375. Accordingly, the method 900 includes an advantage of continuously tracking the guidewire 310 during the placement of the catheter 375. The ability to continuously track the guidewire 310 is attributed to embodiments of the transformer 335 that enable the catheter 375 to be slid over the guidewire 310 without having to move the guidewire 310 a significant distance from the transformer 335.

In other embodiments, systems and method of providing power without contact on the guidewire 310 may be employed to track other instruments. For example, the system 300 may include any variety of instruments instead of, or in addition to, the guidewire 310. In other words, in certain embodiments, the illustrated guidewire 310 may be replaced or supplemented by a drill, a catheter, an endoscope, a laparoscope, a biopsy needle, an ablation device, ultrasound transducers, and flexible ear, nose and throat (ENT) instruments, or other medical devices. In certain embodiments, each of these instruments 130 may include a pickup coil 230 that acts as a secondary winding to the transformer 335 and, thus, receives power via inductive coupling to the transformer 335. The power may be distributed to various tracking components, such as the transmitter coil 330. Further, certain embodiments may include powering and operating the instruments 130 via the power transmitted to the pickup coil 320. Thus, the current induced into the pickup coil 320 may be transmitted to operate devices including an ultrasound transducer, light, light emitting diode, motor, or the like. For example, when using a surgical drill, a distal end of the bit may be used to cut into bone in a patient by rotating the bit via a motor. Disposing a coil in the distal end 370 may enable tracking the location of the cutting, and the motor may be powered via the power supplied via the pickup coil 320. Other embodiments may include any variety of instruments, and any distribution of power via the non-contact powering of the device to a power source.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A tracking system, comprising:
a transformer comprising a primary winding;
a guidewire comprising:
a guidewire body;
a first coil disposed in a first end of the guidewire body; and
a second coil disposed in a second end of the guidewire body, wherein the second coil is inductively coupleable to the primary winding; and
a catheter disposable over the guidewire while the second coil remains inductively coupled to the primary winding.

2. The tracking system of claim 1, wherein the transformer comprises a central axis hole extending though the length of the winding and coincident with a central axis of the coil.

3. The tracking system of claim 2, wherein the primary winding comprises a plurality of windings wrapped completely about the diameter of the central axis hole.

4. The tracking system of claim 2, wherein the primary winding comprises a plurality of windings wrapped around only a portion of the diameter of the central axis hole.

5. The tracking system of claim 1, wherein the primary winding comprises an air gap.

6. The tracking system of claim 1, wherein disposing the guidewire body proximate the transformer inductively couples the second coil to the primary winding.

7. The tracking system of claim 1, wherein the transformer comprises a generally C-shaped core.

8. The tracking system of claim 7, wherein the winding is disposed about a portion of the generally C-shaped core.

9. The tracking system of claim 1, wherein the catheter comprises a hollow tube that is disposed between an inner diameter of the coil and an outer diameter of the guidewire.

10. The tracking system of claim 1, wherein the catheter is configured to slide along the length of the guidewire.

11. The tracking system of claim 1, wherein the guidewire and the winding do not move relative to one another as the catheter slides along the length of the guidewire.

12. The tracking system of claim 1, wherein the second coil remains inductively coupled to the primary winding as the catheter slides along the length of the guidewire.

13. The tracking system of claim 1, wherein powering the first coil is configured to generate a magnetic field.

14. The tracking system of claim 1, comprising tracker electronics.

15. A transformer-coupled guidewire system, comprising:
a coil comprising a primary winding, configured to enable a guidewire including a secondary winding to be inductively coupled to the primary winding, and configured to enable a catheter to be slid over the guidewire while the secondary winding is inductively coupled to the primary winding.

16. The transformer-coupled guidewire system of claim 15, wherein the primary winding comprises a plurality of windings wrapped completely about a central axis hole.

17. The transformer-coupled guidewire system of claim 15, comprising a pot-core.

18. The transformer-coupled guidewire system of claim 15, comprising a generally C-shaped core.

19. The transformer-coupled guidewire system of claim 15, comprising the guidewire disposed proximate to the coil such that the secondary winding is inductively coupled to the primary winding when either of the primary winding or the secondary winding is energized.

20. The transformer-coupled guidewire system of claim 15, wherein the core is electrically and thermally insulated.

21. A tracking method, comprising:
positioning a primary winding about a first coil that is integral to a first end of a guidewire;
inductively coupling the primary winding and the first coil;
transferring the energy between the first coil and a second coil integral to a second end of the guidewire;
passing a catheter over the guidewire; and
tracking the guidewire while the catheter is slid over the guidewire.

22. The tracking method of claim 21, wherein passing the catheter over the guidewire comprises maintaining inductive coupling between the primary winding and the first coil while passing the catheter over the guidewire.

23. An inductively coupled guidewire, comprising:
a primary coil; and
a guidewire mechanically separated from the primary coil and comprising:
a body;
a first coil embedded in a first end of the guidewire; and
a second coil embedded in a second end of the guidewire, the first coil and the second coil being electrically coupled.

24. A tracking system, comprising:
a transformer comprising a primary winding;
a guidewire comprising:
a guidewire body;
a first coil disposed in a first end of the guidewire body; and
a second coil disposed in a second end of the guidewire body, wherein the second coil is inductively coupleable to the primary winding;
a catheter disposable over the guidewire while the second coil remains inductively coupled to the primary winding; and
tracker electronics.
